(19) United States
(12) Patent Application Publication
(10) Pub. No.: US 2011/0135734 A1
(43) Pub. Date: Jun. 9, 2011

(54) Method For the Preparation of Nanoparticles From Nanoemulsions

(75) Inventors: Shlomo Magdassi, Jerusalem (IL); Liat Spernath, Kfar-Saba (IL)

(73) Assignee: YISSUM RESEARCH DEVELOPMENT COMPANY OF THE HEBREW UNIVERSITY OF JERUSALEM, Givat Ram (IL)

(21) Appl. No.: 11/578,945

(22) PCT Filed: Apr. 20, 2005
(86) PCT No.: PCT/IL2005/000416

§ 371 Date: Jan. 11, 2008

Related U.S. Application Data
(60) Provisional application No. 60/563,462, filed on Apr. 20, 2004.

(51) Int. Cl. A61K 9/14; B29B 9/00

(52) U.S. Cl. 424/489; 977/906; 264/5

(57) ABSTRACT

The invention relates to a method for the production of nanoparticles from oil-in-water nanoemulsions, in which the nanoemulsion is prepared by phase inversion techniques. The phase inversion may be achieved by using a constant temperature, where the inversion occurs by continuous addition of water or by varying the temperature involving heating and rapid cooling.

### FIELD OF THE INVENTION

The present invention relates to the formation of nanoparticles, preferably organic nanoparticles, prepared from oil-in-water (O/W) nanoemulsions.

### LIST OF PRIOR ART

The following is a list of prior art which is considered to be pertinent for describing the state of the art in the field of the invention:

U.S. Patent Application publication No. 2003/0206955;

U.S. Pat. No. 6,541,018;

U.S. Pat. No. 6,120,778;

U.S. Pat. No. 6,559,183;

U.S. Pat. No. 3,891,570;

U.S. Pat. No. 4,384,975;

U.S. Pat. No. 5,407,609;

U.S. Pat. No. 5,705,196;

Desgouilles et al. Langmuir, 19:9504-9510, (2003);

Antonietti et al. Prog. Polym. Sci. 27:689-757, (2002).

### BACKGROUND OF THE INVENTION

Nanoemulsions are a class of transparent or translucent emulsions, having a droplet size range between 40-500 nm. Unlike microemulsion, nanoemulsions are only kinetically stable. However, the long-term physical stability of nanoemulsions is excellent, compared to macroemulsions.

Nanoemulsions are used for various applications such as reaction media for polymerization, personal care and cosmetics, health care and agrochemicals. Using nanoemulsion in industrial applications is very attractive due to several reasons, including, inter alia, the following:
1. The very small droplet size prevents creaming or sedimantation.
2. The small droplet size and hence the large surface area makes these systems suitable for efficient delivery of active components.
3. Nanoemulsions do not require high concentration of surfactants as typically used with microemulsions. These systems can be prepared using moderate surfactant concentrations (between 4-8% wt %).

The nanometric size of the oil droplet of the emulsion is usually achieved by applying high shear forces (high input of mechanical energy). U.S. Pat. No. 6,541,018, U.S. Pat. Nos. 6,120,778 and 6,559,183 and U.S. Patent Application No. 2003/0206955 disclose processes of preparing nanoemulsions using a high pressure homogenizer.

Nanoparticles can be obtained from confined nanometeric structures, such as nanoemulsion droplets. For example, nanoemulsions that contain as the dispersed phase a water immiscible solvent and a dissolved active substance. Nanoparticles of the active substance can be obtained upon removal of the solvent by evaporation or extraction. Yet, the oil droplets of the nanoemulsions can be used as nanoreactors for chemical reactions such as polymerizations, resulting in polymeric nanoparticles.

Several methods for preparing microparticles and nanoparticles are described in the art. Examples of such processes are: emulsion-solvent evaporation, emulsion-solvent extraction, anti-solvent precipitation, emulsion polymerization and miniemulsion polymerization.

The emulsion solvent evaporation method is well described by Desgouilles et al [Desgouilles et a., 2003 ibid]. This method is based on the emulsification of an organic solution of an active substance in an aqueous phase by using a high pressure homogenizer (microfluidizer) followed by the evaporation of the organic solvent under reduced pressure or vacuum. The evaporation process leads to the precipitation of the active substance as nanoparticles.

U.S. Pat. Nos. 3,891,570 and 4,384,975 disclose preparation of microparticles by evaporation of an organic solvent from an emulsion.

Until now, formation of nanoparticles from emulsions was possible only if the emulsion droplets are in the nanometric size range, which was achieved previously by applying high shear forces to the crude emulsion, by equipment such as high pressure homogenizers or by applying high ultrasound energy. The high shear homogenizers are very costly, and their use introduces various production problems, such as low production rate, elevated temperatures at the homogenization chamber (which may be detrimental to heat and pressure sensitive materials), possible clogging of the homogenizer orifices, and possible wear of the sealing rings and incompatibility of the sealing materials with many solvents.

U.S. Pat. No. 5,407,609 discloses a microencapsulation process based on solvent extraction. The process involves preparation of an emulsion composed of an active substance and a solvent as the dispersed phase. In the second step the emulsion is added to an extraction medium that extracts the solvent from the droplets resulting in a microencapsulated product.

U.S. Pat. No. 5,705,196 discloses an anti-solvent precipitation process in which an active substance is dissolved in an organic solvent that has a water miscibility of more than 10% (for example: acetone). When the organic solution comes in contact with water the organic solvent rapidly diffuses to the aqueous phase which causes immediate precipitation of the active substance. No emulsion is obtained in this process.

The emulsion polymerization process is described in a review article by Antonietti et al. [Antonietti et al. 2002, ibid.]. In this process the polymerization starts from a situation where large monomer droplets stabilized by surfactant molecules and empty or monomer-swollen surfactant micelles coexist. The water-soluble initiator forms oligoradicals with the slightly water-soluble monomer units, until the oligoradicals are hydrophobic enough either to enter the micelles or to nucleate particles from the continuous phase. During the polymerization the monomer diffuses from the larger monomer droplets through the water phase to the micelles in order to sustain polymer particle growth until the monomer droplets have vanished. Particles with a diameter of usually larger than 100 nm are formed.

### SUMMARY OF THE INVENTION

The present invention is based on the finding that it is possible to produce nanoparticles of active agents (preferably organic active ingredients) from emulsions using phase inversion techniques that convert the emulsions from one form (water-in-oil/oil-in-water) to the other form (oil-in water/water in oil). The conversion may be achieved by using a constant temperature, where the conversion occurs by the continuous addition of water, causing a phase inversion, or by increase of temperature causing said phase inversion. In accordance with the methods of the invention there requirement for high shear force in eliminated.

Constant Temperature Aspect of the Invention

According to a first embodiment of the constant temperature aspect of the present invention there is provided a method for the production of nanoparticles of an active agent, the method comprising:
(a) mixing the active agent with a volatile solvent and at least one non ionic surfactant;
(b) adding to the mixture of (a) an aqueous phase to form a water-in-oil emulsion;
(c) continuously adding to the emulsion of (b) water at a rate enabling phase inversion and formation of oil-in-water nanoemulsion;
(d) evaporating the volatile solvent from the oil-in-water nanoemulsion of (c) to obtain nanoparticles of the active ingredient.

According to a second embodiment of the constant temperature aspect of the present invention there is provided a method for the production of nanoparticles of an active agent, the method comprising:
(a) mixing the active agent with liquid monomers capable of polymerizing, and at least one non ionic surfactant;
(b) adding to the mixture of (a) an aqueous phase to form a water-in-oil emulsion;
(c) continuously adding to the emulsion of (b) water at a rate enabling phase inversion and formation of oil-in-water nanoemulsion;
(d) applying conditions enabling polymerization of the liquid monomer in the oil-in-water nanoemulsion of (c) to obtain nanoparticles of the active ingredient.

For purpose of convenience in the description below the term “constant temperature aspect” will be used to refer collectively to the two embodiments (first and second embodiment described above).

Phase Inversion Temperature Aspect of the Invention

According to a first embodiment of the phase inversion temperature aspect of the present invention there is provided a method for the production of nanoparticles of an active agent, the method comprising:
(a) mixing the active agent with a volatile solvent, at least one nonionic surfactant and an aqueous phase, to obtain a crude oil-in-water emulsion;
(b) raising the temperature of the crude oil-in-water emulsion of (a) to a phase inversion temperature (PIT) to obtain, a water-in-oil emulsion;
(c) cooling the water-in-oil emulsion of step (b) to obtain an oil-in-water nanoemulsion;
(d) evaporating volatile solvent from the oil in water nanoemulsion of (c) at a temperature below the PIT, to obtain nanoparticles of the active ingredient.

According to second embodiment of the phase inversion temperature aspect of the present invention there is provided a method for the production of nanoparticles of an active ingredient, the method comprising:
(a) mixing the active agent with liquid monomers capable of polymerizing, at least one nonionic surfactant and an aqueous phase to obtain a crude oil-in-water emulsion;
(b) raising the temperature of the crude oil-in-water emulsion of (a) to a phase inversion temperature (PIT) to obtain, a water-in-oil emulsion;
(c) cooling the water-in-oil emulsion of step (b) to obtain an oil-in-water nanoemulsion;
(d) providing conditions enabling polymerization, and which do not cause a raise of a temperature to a temperature above the PIT temperature, to the oil-in-water nanoemulsion of (c) to obtain nanoparticles of the active ingredient.

For purpose of convenience in the description below the term “phase inversion temperature aspect” will be used to refer collectively to the two embodiments (first and second embodiment described above).

The nanoparticles of the present invention are preferably organic nanoparticles and preferably hydrophobic (i.e. the active agent of the nanoparticles is preferably an organic active agent and is preferably hydrophobic).

As used herein by the term “aqueous phase” (in the constant temperature aspect and phase inversion temperature aspect) is meant aqueous liquid (such as water or aqueous solution).

As used herein by the term “water” (in step (c) of the constant temperature aspect) is meant also an aqueous liquid (such as water or aqueous solution).

As used herein the term “liquid monomers” refers to liquid comprising of molecules which may be polymerized to form a polymer or copolymer.

As used herein by the term “crude emulsion” is meant that the droplets size of the emulsion is not in the nanoscale range.

As used herein by the term “continuously adding water” (in step (c) in the constant temperature aspect) in meant gradual addition of water.

### DESCRIPTION OF FIGURES

FIG. 1 displays conductivity change during the formation of nanoemulsions with toluene according to one embodiment of the invention;

FIG. 2 shows a Scanning Electron Microscope (SEM) image of Ethyl-cellulose nanoparticles obtained to another embodiment of the invention.

FIG. 3 displays conductivity change during a heating process involved in the formation of nanoemulsions comprising lauryl acrylate and Decaethylene glycol oleyl ether (Brij 96v) according to yet another embodiment of the invention.

FIG. 4 show a SEM photo of poly-lauryl acrylate nanoparticles obtained from the nanoemulsions of FIG. 3;

FIG. 5 shows an Atomic Force Microscope (AFM) image of the poly-lauryl acrylate nanoparticles shown in FIG. 4.

### DETAILED DESCRIPTION OF THE INVENTION

The present invention describes a simple and low cost method for formation of organic nanoparticles from oil-in-water nanoemulsions, in which the nanoemulsion is prepared by inversion techniques, without the use of high shear forces instruments.

The object of this invention is to prepare nanoparticles (preferably organic nanoparticles) from nanoemulsions, which are prepared by phase inversion techniques, without the need for high shear equipment. In accordance with the invention the droplets of the nanoemulsions or the content of the droplets (which are composed of a water immiscible liquid and dissolved materials) are converted into nanoparticles.

The nanoparticles in this invention are prepared from nanoemulsions which are prepared using phase inversion techniques.

By one embodiment of the invention (hereinafter “constant temperature aspect”) phase inversion occurs at constant temperature.

By another embodiment of the invention (hereinafter “PIT aspect”) the phase inversion occurs by varying the temperature and involves heating and rapid cooling.

As used herein the term “volatile solvent” refers to a solvent having an evaporation rate higher than water.

The volatile solvent is part of the oil phase of the emulsion and is preferably immiscible with water. The volatile solvent may be slightly soluble in water (for example toluene having a solubility in water of 0.05 gr/100 ml). The volatile solvent is a hydrophobic organic solvent.

As used herein the term “active agent” refers to any molecule or substance that can be used in agriculture, industry (for example polymer industry), medicine, cosmetics and which grants the final product (pesticide, drug, cosmetics etc.) at least one desired property.

The active agent (active ingredient) is preferably an organic hydrophobic ingredient. Preferably the active agent is water insoluble. Preferably the active agent is soluble in the volatile solvent (i.e. the active agent is dissolved in the volatile solvent (oil phase) of the emulsion).

### Constant Temperature Aspect

In accordance with one embodiment of the constant temperature aspect, using volatile solvents, the present invention concerns a method for the production of nanoparticles of an active agent, the method comprising:
(a) mixing the active agent with a volatile solvent and at least one non ionic surfactant;
(b) adding to the mixture of (a) an aqueous phase to form a water-in-oil emulsion;
(c) continuously adding to the emulsion of (b) water at a rate enabling phase inversion and formation of oil-in-water nanoemulsion;
(d) evaporating the volatile solvent from the oil-in-water nanoemulsion of (c) to obtain nanoparticles of the active ingredient.

Preferably the volatile solvent is selected from toluene, butyl acetate, propyl acetate, hexane, cyclohexane, carbon tetrachloride, trichloroethane, trichloromethane, 2-butanol, methyl acetate, heptane, methylpropionate, propyl acetate and any mixture of the above. More preferably the volatile solvent is toluene or butyl acetate.

The mixture of step (a) should contain at least one non ionic surfactant, but may contain two or more non ionic surfactants, or one or more non ionic surfactant and an additional ionic surfactant such as the anionic surfactant SDS.

The non ionic surfactant in accordance with this aspect of the invention should preferably have a high HLB (Hydrophilic Lypophilic Balance) value.

Preferably the non-ionic surfactant in step (a) have HLB value in the range of 10-20.

Preferably the non-ionic surfactant is a hydrophilic non-ionic surfactant.

Preferably the surfactant is capable of dehydration and most preferably the non ionic surfactant comprising ethylene oxide groups.

Preferably the non-ionic surfactant is selected from polyethoxylated sorbitan esters (such as tween 20, 40, 60 or 80), polyglycerol esters (such as decaglycerolmonolaurate, decaglycerol monostearate, decaglycerol monooleate), sucrose esters, ethoxylated alcohols (such as Brij 96V) and octylphenol ethoxylated (such as Triron X surfactant series) and mixtures of any of the above.

The non-ionic surfactant may further comprise sorbitan esters (such as span 20, 40, 60 or 80).

When sorbitan esters (which is a hydrophobic non-ionic surfactant (having a low HLB value)) is used, it is not used alone but in combination with a hydrophilic non-ionic surfactant (such as mentioned above).

Preferably the non ionic surfactant comprises a mixture of polyethoxylated sorbitan esters with a surfactant selected from polyglycerol esters, sucrose esters, sorbitan esters. Preferably the non-ionic surfactant comprises a mixture of polyethoxylated sorbitan esters with sorbitan esters.

The non-ionic surfactant may also be mixtures of polyglycerol esters and sorbitan esters, or mixtures of sucrose esters and sorbitan esters.

Preferably when the mixture in step (a) comprises a mixture of surfactants having a low and high HLB (for example a mixture of hydrophobic and hydrophilic surfactants), the HLB of the mixture of surfactants is preferably in the range 10-20.

The mixture of step (a) may further comprise an additional ionic surfactant.

More preferably the ionic surfactant is sodium dodecyl sulphate (SDS).

The surfactants and HLB values described above may be used in the constant temperature aspect and phase inversion temperature aspect of the present invention.

Optionally, the mixture of step (a) may further comprise an ingredient selected from a polymer, a cosurfactant, a cosolvent, and combinations of any of the above. (To aid in the formation of the nanoparticles).

Preferably the polymer is selected from ethyl cellulose, propyl hydroxyl ethyl cellulose, polysterene, polymethylmethacrylate, polyvinyl butyral, and mixtures of any of the above. More preferably the polymer is ethyl cellulose.

Preferably the polymer is a hydrophobic polymer. Preferably the polymer is soluble in the volatile solvent.

The cosolvent may be a C₅-C₁₈ alcohol.

The cosolvent may be for example pentanol, octanol, decanol, dodecanol, or mixtures of any of the above.

The addition of water in step (c) and preferably also in step (b) of the method should be a gradual addition enabling first the formation of water-in-oil emulsion and then the phase inversion to oil-in water emulsion, for example at a rate of 0.1-1 ml/minute, preferably at a rate of 0.5 ml/minute. However the man versed in the art will appreciate that the rate may depend on many factors such as the size of the sample, the temperature, the specific compounds used and may adjust after minimal trial and error experiments, the rate to such enabling easy control of the phase conversion.

The method may further comprise an additional step after step (d) selected from spray-drying or lyophilization thereby forming a powder of nanoparticles.

Alternatively, in step (d) evaporation may be carried out simultaneously with spray-drying or lyophilization converting the nanoparticles formed by evaporation into powder of nanoparticles. According to this alternative, evaporation may take place during spray-drying or lyophilization processes, so that in a single step the evaporation of the volatile solvent and simultaneous conversion of the nanoparticles into powder of nanoparticles takes place.

In accordance with another embodiment of the constant temperature aspect, suitable for liquid monomers, the present invention concerns a method for the production of nanoparticles of an active agent, the method comprising:
(a) mixing the active agent with liquid monomers capable of polymerizing, and at least one non ionic surfactant;
(b) adding to the mixture of (a) an aqueous phase to form a water-in-oil emulsion;
(c) continuously adding to the emulsion of (b) water at a rate enabling phase inversion and formation of oil-in-water nanoemulsion;
(d) applying conditions enabling polymerization of the liquid monomer in the oil-in-water nanoemulsion of (c) to obtain nanoparticles of the active ingredient.

Preferably the monomers are selected from sterene, lauryl acrylate, stearyl acrylate, isodecyl acrylate, isooctyl acrylate, isotridecyl acrylate, isobornyl acrylate, lauryl methacrylate, lauryl methacrylate, stearyl methacrylate, isobornylmethacrylate, and mixtures of any of the above. Most preferably the monomer is lauryl acrylate.

The non-ionic surfactant, surfactants, surfactant mixtures and preferred HLB values may be as described above in the constant temperature aspect, using volatile solvents.

Preferably the non-ionic surfactant is selected from ethoxylated alcohols, polyethoxylated sorbitan esters, polyglycerol esters, sucrose esters, and mixtures of any of the above.

The non-ionic surfactant may further comprise sorbitan esters (such as span).

Additional examples of surfactants are described above in the constant temperature aspect of the invention.

Preferably the nonionic surfactant in step (a) have a HLB value in the range of 10-20.

For polymerization purposes, an initiator should be added either to the mixture of step (a), or immediately prior to step (d).

The method further comprises adding an initiator to the mixture of step (a) or immediately prior to step (d).

The initiator may be for example a thermal initiator, or a UV-activated initiator.

Preferably the thermal initiator is selected from ammonium persulfate, potassium persulfate, lauryl peroxide, benzoyl peroxide, 2,2-azodiisobutyrodinitrile, and mixtures of any of the above.

The “conditions” stipulated in step (d), should be either applying suitable temperature, or applying UV radiation in accordance with the nature of the initiator.

Preferably the initiator is a thermal initiator and the condition in step (d) is applying suitable temperatures.

Preferably the initiator is a UV activated initiator and the condition in step (d) is applying UV radiation.

Preferably the thermal initiator is water soluble thermal initiator and is added in step (c). Preferably the water soluble initiator is ammonium persulphate.

Preferably the UV activated initiator is selected from 2-Hydroxy-2-Methyl Propiophenone (for example Daracure 1173),-2-methyl-1-[4-(methylthio)phenyl]-2-(4-morpholinyl)-1-propanone (for example Irgacure 907), 1-hydroxy-cyclohexyl-phenyl-ketone (for example SarCure SR1122), 2-hydroxy-2-methyl-1-phenyl propanone (for example SarCure SR 121), ethyl 4-(dimethylamino)benzoate (for example SarCure SRI 125) isopropylthioxanthone (for example Quantacure ITX), and mixtures of any of the above.

An initiator (thermal or UV activated) may be hydrophobic and in such a case may be added to the oil phase (added in step (a)) or may be hydrophilic and be added to the water phase, (immediately prior to step (d)).

Preferably the conditions in step (d) are selected from applying suitable temperature and applying UV radiation.

The method may further comprise adding an activator in step (a).

The method may further comprise adding an activator immediately prior to step (d).

In such a case where an activator is added in step (a) or immediately prior to step (d) the polymerization is preferably carried at 20-40° C., more preferably 40° C.

As described above, it is possible also optionally to add an activator either in step (a), or immediately prior to step (d), in order to enable polymerization under more favorable conditions, for example, at room temperature conditions.

The activator, which addition is merely optional, may again be added to the oil phase in step (a) (if hydrophobic), or to the water phase immediately prior to step (d) (if hydrophilic).

As in accordance with the prior embodiment, immediately after polymerization, the particles may be converted into powder of nanoparticles by spray drying or lyophilization. (in this case, the method may further comprise an additional step after step (d) selected from spray-drying or lyophilization thereby forming a powder of nanoparticles.)

Obviously, the polymeric nanoparticles can be formed using a polymer only, meaning that the nanoparticles are composed of polymer only. In such a case the active agent is a polymer.

The term “nanoparticles” in accordance with this polymerization embodiment, may also refer to nanocapsules, encapsulating the active agent in a core-shell structure), wherein the polymerization takes place in the interface between the oil and water.

In that case, two different monomers are used, a first monomer being in the oil phase added in step (a), and a second monomer (dissolved in the aqueous phase) is added in steps (b) and (c), and nanoencapsulation takes place in the interface between the first and second monomers, during their polymerization. (i.e. nanoencapsulation takes place in the interface between the oil droplets and the continuous aqueous phase). The second monomer may also be added (preferably stepwise) after nanoemulaion is prepared, in conditions favoring interfacial polymerization reaction, and nanoencapsulation takes place at the interface between the first and second monomers, during their polymerization.

According to the constant temperature aspect of the present invention the phase inversion is conducted at a constant temperature, preferably in the range 20-40° C., and more preferably at room temperature (20-25° C.).

The phase inversion at constant temperature involves preparing the nanoemulsions by gradual addition (for example 0.5 ml/min) of water to a mixture of oil and non-ionic surfactant/s. The emulsions are prepared at room temperature, with continuous stirring, using for example a magnetic stirrer. At the beginning of the process when there is a low content of water, a water-in-oil (W/O) emulsion is formed. At the end of the process, when there is high percentage of water in the system, water becomes the continuous phase and an oil-in-water (O/W) emulsion is formed. The phase inversion occurs due to the high water/oil ratio and the high HLB value of the surfactant or surfactant mixture, which favors the formation of an oil-in-water emulsions.

The emulsions formed using this process have a mean droplet sizes between 40-500 nm. The nanometric droplet size of the emulsion is a result of phase transitions that occur during the gradual addition of the water. The phase transitions include a microemulsion phase and formation of liquid crystals (Solans et al. Langmuir, 17:2076-2083, 2001).

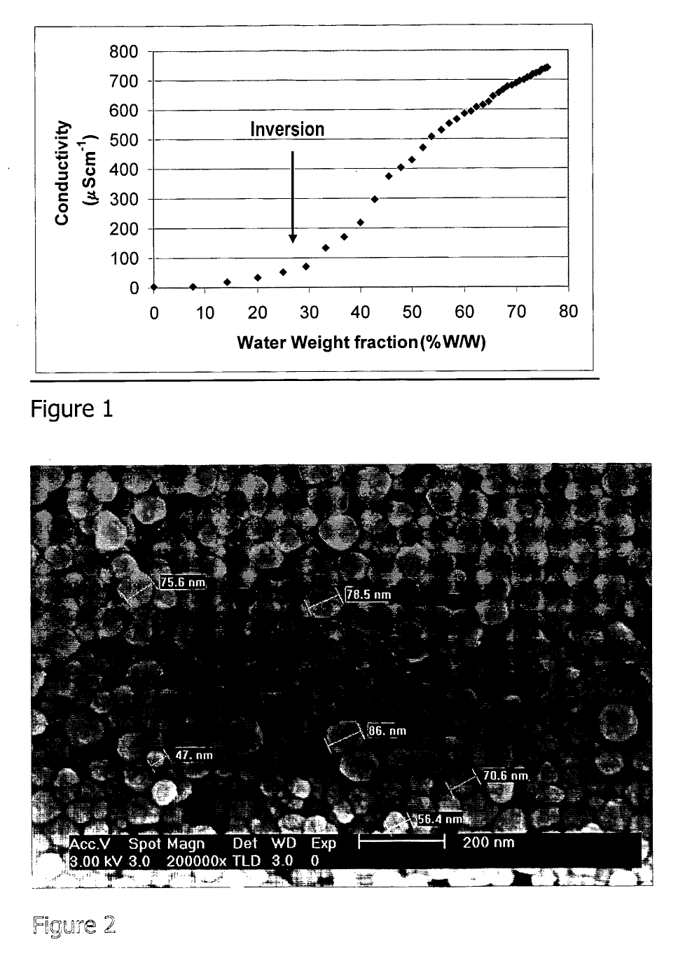

One can find the inversion point of the system by replacing water with 10 mM NaCl solution and measuring the conductivity of the system, which increases during the process as water becomes the continuous phase, as shown for example in FIG. 1 below.

The nanoemulsions of this type which are described in the literature and are composed of non-volatile, hydrophobic oils such as Decane. There are no reports yet on formation of nanoemulsions containing volatile solvents, using this method, nor any mention of the use of the nanoemulsions for the formation of nanoparticles or nanocapsules.

We found, surprisingly, that if we prepare the nanoemulsions with volatile solvent in which active agents like: drugs, pigments, pesticides, antioxidants and preservatives are dissolved it is possible to form nanoparticles, after evaporation of the solvent.

Examples of non-ionic surfactants used in this process are: Sorbitan esters, polyethoxylated sorbitan esters and glycerol esters. Optionally when in addition to the at least one non-ionic surfactant an ionic surfactant is used, it is possible to use anionic surfactants such as SDS.

The solvents used in this invention are volatile solvents like toluene and butyl acetate.

For example, formation of organic particles such as ethyl cellulose is possible, if the nanoemulsion is prepared while the ethyl cellulose is dissolved prior to emulsification, in the toluene. Than, after performing the inversion and obtaining the nanoemulsion, the toluene evaporates under reduced pressure or increased temperature, or a combination of reduced pressure and increased temperature, and ethyl cellulose nanoparticles are obtained as a dispersion in water. The dispersion can be further converted into a powder of nanoparticles, for example by spray drying or lyophilization.

Obviously, one can dissolve many other water insoluble active agents in the solvent phase, thus obtaining nanoparticles of the desired molecules. The nanoparticles can be composed of one type of molecule, or a mixture of several different types of molecules, for example polymeric particles in which a drug, a colorant, or any other active molecule is dispersed.

The surfactants are preferably used in a concentration of about 1-10% w/w, most preferably about 5% w/w (based on the total weight of the emulsion).

The oil is preferably used in a concentration of about 1-50% w/w, most preferably about 20% w/w (based on the total weight of the emulsion).

### The PIT Aspect of the Invention

Another method of preparing nanoparticles from nanoemulsions without high shear equipment is by using the phase inversion temperature (PIT) technique. In this technique, first a crude oil in water (O/W) emulsion is prepared by simple stirring or by using a conventional homogenizer such as Ultra-Turrax homogenizer. Then, the crude emulsion is heated to a specific temperature (the phase inversion temperature), in which a water-in -oil (W/O) emulsion is formed. The hot emulsion is than cooled rapidly in an ice bath, resulting in a nanoemulsion.

The phase inversion temperature (PIT) technique makes use of the sensitivity to temperature of O/W emulsions stabilized by nonionic surfactants. Surfactants that contain ethoxylated groups, undergo a dehydration process during heating and become more hydrophobic (oil soluble), thus favoring the formation of a water-in-oil (W/O) emulsions.

During the heating process these emulsions pass through a microemulsion phase wherein an ultra-low interfacial tension between the oil and aqueous phases exists. The microemulsion phase allows finely dispersed O/W emulsions to be produced upon cooling without a high input of mechanical energy. The result of the PIT process is nanoemulsions having a droplet size of 70-200 nm (Forester et al. Advances in Colloid and Interface Science, 58:119-149, 1995). Typically, oils used in the literature in this technique are: mineral oils, decyl oleate, 2-octyl dodecanol and isopropyl myristate.

One can find the phase inversion point by measuring the conductivity of the system during heating using 10 mM NaCl as the aqueous phase. The conductivity of the system increases during the heating process, due to higher mobilty of the ions, and decreases sharply at the inversion point, when oil becomes the continuous phase.

Thus, in accordance with one embodiment of the PIT aspect of the invention suitable for volatile solvents, the present invention concerns a method for the production of nanoparticles of an active agent, the method comprising:
(a) mixing the active agent with a volatile solvent, at least one nonionic surfactant and an aqueous phase, to obtain a crude oil-in-water emulsion;
(b) raising the temperature of the crude oil-in-water emulsion of (a) to a phase inversion temperature (PIT) to obtain, a water-in-oil emulsion;
(c) cooling the water-in-oil emulsion of step (b) to obtain an oil-in-water nanoemulsion;
(d) evaporating volatile solvent from the oil in water nanoemulsion of (c) at a temperature below the PIT, to obtain nanoparticles of the active ingredient.

The volatile solvent may be as described above in the constant temperature aspect of the invention. More preferably the volatile solvent is selected from toluene, butyl acetate, and any mixture of the above.

The non ionic surfactant may be as described above in the constant temperature aspect of the invention.

Preferably the non-ionic surfactant in step (a) have HLB value in the range of 10-20.

Preferably the non ionic surfactant comprising ethylene oxide groups.

Preferably the non-ionic surfactant is selected from polyethoxylated sorbitan esters, polyglycerol esters, sucrose esters, ethoxylated alcohols, octylphenol ethoxylated, and mixtures of any of the above.

The mixture of step (a) may further comprise an additional ionic surfactant.

Preferably the ionic surfactant is an anionic surfactant such as sodium dodecyl sulphate.

The mixture of step (a) should contain at least one non ionic surfactant, but may contain two or more non ionic surfactants, or one or more non ionic surfactants and an additional ionic surfactant.

The surfactant may be present in the volatile solvent, or in the aqueous phase of, where more than one surfactant is used, some may be present in the solvent and some in the aqueous phase.

Optionally, the mixture of (a) may further comprise an ingredient selected from a polymer, a cosurfactant, a cosolvent, and mixtures of any of the above. (These ingredients may aid the formation of the nanoparticles).

The polymer, cosurfactant and cosolvents may be as described above under the constant temperature aspect.

Preferably the raise to the PIT temperature, in step (b), is gradual. As indicated above, the exact PIT temperature may be determined by using the NaCl in the aqueous phase and monitoring the conductivity of the system.

Preferably the cooling in step (c) is rapid cooling in order to stabilize the nanoemulsion obtained during the inversion.

Preferably the “cooling” is to a temperature of 0-10° C., more preferably to about 4° C.

The evaporation in step (d) should be done at a temperature below the PIT, (such as for example by using low pressure conditions).

Optionally, the method may further comprise an additional step after step (d) selected from spray drying or lyophilization thereby forming a powder of nanoparticles. (In this additional step nanoparticles may be converted into powder nanoparticles by spray drying or lyolphilization).

Alternatively, evaporation in step (d) may be carried out simultaneously with spray drying or lyophilization converting the nanoparticles formed by evaporation into powder of nanoparticles. (In this case evaporation may take place as a single step occurring during the spray drying or lyophilization.) In accordance with another embodiment of the PIT aspect of the invention suitable where the solvent is liquid monomers, the present invention concerns a method for the production of nanoparticles of an active ingredient, the method comprising:
(a) mixing the active agent with liquid monomers capable of polymerizing, at least one nonionic surfactant and an aqueous phase to obtain a crude oil-in-water emulsion;
(b) raising the temperature of the crude oil-in-water emulsion of (a) to a phase inversion temperature (PIT) to obtain, a water-in-oil emulsion;
(c) cooling the water-in-oil emulsion of step (b) to obtain an oil-in-water nanoemulsion;
(d) providing conditions enabling polymerization, and which do not cause a raise of a temperature to a temperature above the PIT temperature, to the oil-in-water nanoemulsion of (c) to obtain nanoparticles of the active ingredient.

Preferably the monomers are selected from sterene, lauryl acrylate, stearyl acrylate, isodecyl acrylate, isooctyl acrylate, isotridecyl acrylate, isobornyl acrylate, lauryl methacrylate, lauryl methacrylate, stearyl methacrylate, isobornylmethacrylate, and mixtures of any of the above. Most preferably the monomer is lauryl acrylate.

The surfactants and preferred HLB values may be as described above under constant temperature aspect.

Preferably the non-ionic surfactant is selected from polyethoxylated sorbitan esters, polyglycerol esters, sucrose esters, ethoxylated alcohols, octylphenol ethoxylated, and mixtures of any of the above.

Additional examples of surfactants are as described in the constant temperature aspect.

Preferably the nonionic surfactant in step (a) have HLB value in the range of 10-20.

The method further comprises adding an initiator.

In order to achieve polymerization a polymerization initiator should be added to the system. The initiator may be a thermal initiator or a UV activated initiator.

Preferably the UV activated initiator is hydrophilic and is added to the water in the oil-in-water nanoemulsion of step (c).

Preferably the initiator is UV initiator and the condition of step (d) is application of UV radiation sufficient to begin polymerization.

The polymerization, occurring in step (d) should take place at a temperature below PIT so that the phase inversion process is not “undone” i.e. so that the emulsion does not undergo an additional phase conversion (and in order to retain the emulsion structure (the nano-droplets)).

Therefore if a thermal initiator is used it should be added after the phase inversion raise of the temperature in step (b) (preferably after cooling in step (c)), for example, between steps (c) and (d), and then the condition of step (d) is to raise the temperature again (after the cooling of step (c)) to initiate polymerization, but this raise of step (d) should be to a temperature lower than PIT temperature ( this may be achieved for example, by the use of an activator which causes polymerization at lower temperatures as will be explained below).

In such a case, the thermally activated initiator should be hydrophilic, as it should be added to the water phase of the oil-in-water nanoemulsion obtained by step (c).

By another alternative, the initiator is activated by UV application (UV activated initiator (photoinitiator)), and in such a case, if it is hydrophobic, it may be added to the oil phase of the mixture in step (a), or alternatively if it is hydrophilic, it may be added to the aqueous phase in step (a) or to the water in the oil-in-water nanoemulsion of step (c). Where a UV initiator is used, the condition of step (d) is application of UV radiation sufficient to begin polymerization.

The method may further comprise adding an activator.

In order to enable polymerization under more convenient conditions (for example at room temperature) an activator may optionally be added. The activator may be added in step (a) either to the solvent (oil phase) or the aqueous phase, or alternatively in step (c), for example into the water phase.

The thermal activator may be for example transition metal ions such as Fe⁺², Co⁺², Cu⁺¹, preferably Fe⁺².

The term “nanoparticles” in accordance with this polymerization embodiment, may also refer to nanocapsules, encapsulating the active agent in a core-shell structure), wherein the polymerization takes place in the interface between the oil and water.

In that case, two different monomers are used, one monomer being in the oil phase added in step (a), and another monomer dissolved in the aqueous phase added in steps (b) and (c), and nanoencapsulation takes place in the interface between the two monomers, during their polymerization [in accordance with the constant temperature aspect].

In accordance with the PIT aspect, two different monomers may be used, a first monomer is being in the oil phase (the first monomer can be the oil phase itself) and a second monomer is dissolved in the aqueous phase, both said first and second monomers are added in step (a) and nanoencapsulation takes place in the interface between the first and second monomers during their polymerization. (i.e. nanoencapsulation takes place in the interface between the oil droplets and the continuous aqueous phase).

The “oil phases” (in accordance with the “PIT aspect”) may be non-volatile monomers such as acrylates, which can undergo polymerization, or volatile solvents (containing dissolved materials) such as toluene or butyl acetate, which can be removed by evaporation, resulting in the formation of nanoparticles. Both steps (polymerization or evaporation) are carried out after the inversion of the emulsions had occurred (after the nanoemulsions are obtained). The nanoparticles formation (either by solvent evaporation or by polymerization) has to occur at temperatures lower than the PIT in order to retain the droplets identity.

Nanoemulsion polymerization may be carried out using a water soluble thermal initiator (added in step (c)) like Ammonium persulfate and a water soluble activator like FeSO₄ (Fe⁺² ions) (added in step (a) or after step (c) more preferably after step (c)). In addition styrene should be added to the aqueous phase in order to form hydrophobic radicals, having the ability to enter the oil droplets. The optional addition of the activator enables the polymerization to occur at temperature lower than the PIT, and therefore there is no danger of damaging the nanoemulsions due to heating of the emulsion above or close to the phase inversion temperature.

Another option of polymerization of the oil droplets is to use UV initiator and apply UV radiation in step (d). That can be performed by addition of photoinitiators to the oil phase (in step (a)), or photoinitiator to the aqueous phase (in step (a) or after step (c)) and exposure to UV radiation after the inversion of the emulsion has occurred.

When using a volatile solvent it is important to adjust the evaporation temperature to be lower than the PIT. This can be done by evaporation that takes place at low pressure conditions ensuring that evaporation occurs at a temperature lower than the PIT.

An example of a monomers used in this process is lauryl acrylate. The monomer is preferably used in a concentration of 10-50%w/w based on the final emulsion composition, more preferably about 20% w/w based on the final emulsion composition. The phase inversion temperature of the lauryl acrylate emulsions prepared with surfactant such as Brij 96 is between 50° C.-70° C. (depending on surfactant concentration, surfactant type and type and concentration of co-surfactant, if added).

The surfactants used in this process are surfactants that can undergo dehydration preferably ethoxylated alcohols like Brij surfactants. Optionally, a long chain alcohol, like octanol can be added as a co-surfactant. The surfactants are preferably used in a concentration between 3-7% w/w based on the final emulsion composition, more preferably 5-7%w/w (based on the final emulsion composition). The alcohol is preferably used in a concentration between 0-10%.

One can control the droplet size and the PIT by a selection of a surfactant or surfactants, co-surfactant and their concentration, or by the selection of the oil or mixture of oils.

### DESCRIPTION OF SPECIFIC EXAMPLES

The concentration values (%) described in the examples below are in w/w.

### Phase Inversion at Constant Temperature:

### Example 1

### Formation of Nanoemulsion with Toluene

A nanoemulsion containing toluene as the oil phase stabilized by 5% Span 20 and Tween 20 (HLB-14) as the surfactants was prepared with the following composition:

20% Toluene

3.3% Tween 20

1.7% Span 20

76% 10 mM NaCl

Specifically, the above amounts of Tween 20, Span 20 and toluene were mixed. The aqueous phase (10 MM NaCl) was added gradually (0.5 ml/min). The addition of the aqueous phase was done under continuous stirring. A nanoemulsion was formed having an average droplet size of 237 nm. FIG. 1 displays the conductivity change of the system during the process.

### Example 2

### Formation of Nanoemulsion with Toluene

A nanoemulsion containing toluene as the oil phase stabilized by 5% Decaglycerol monolaurate and Span 20 (HLB-14) as the surfactants was prepared, using the following composition:

20% Toluene

4.35% Decaglycerol monolaurate

0.65% Span 20

75% 10 mM NaCl

Specifically, the above amount of decaglycerol monolaurate, Span 20 and toluene were mixed. The aqueous phase (10 mM NaCl) was added in the same manner as described in example 1. The conductivity change of the system was monitored as described in example 1. The result of the process is an emulsion having an average droplet size of 183 nm.

### Example 3

### Formation of Ethyl Cellulose Nanoparticles

Ethyl cellulose nanoparticles having the following composition were also prepared, suing the following specific composition.

### Composition.

19.2% Toluene

0.8% Ethyl cellulose (Ethyl cellulose from Sigma, having a viscosity of a 5% solution in toluene:ethanol 4:1 at 25° C.˜45 cPs)

4.35% Decaglycerol monolaurate

0.65% Span 20

75% 10 mM NaCl

At first stage, ethyl cellulose was dissolved in toluene. Decaglycerol monolaurate and Span 20 were added to the solution. The aqueous phase (10 mM NaCl) was added in the same manner as described in Example 1. The conductivity change of the system was monitored as described in Example 1. A nanoemulsion was formed comprising ethyl cellulose dissolved in toluene as the dispersed oil phase. The average droplet size of the emulsion was 200 nm.

Toluene was evaporated using a rotor stator evaporator, under reduced pressure, at 40° C., resulting in the formation of ethyl cellulose dispersion. The average size of the ethyl cellulose nanoparticles was 162 nm.

FIG. 2 shows a SEM image of Ethyl-cellulose nanoparticles obtained by the above method.

### Example 4

### Nanoparticles of Ethyl Cellulose

In this example a higher concentration of ethyl cellulose was used, to obtain nanoparticles using the following specific composition:

### Composition.

18% Toluene

2% Ethyl cellulose (Ethyl cellulose from Sigma, having a viscosity of a 5% solution in toluene:ethanol 4:1 at 25° C.˜45 cPs).

4.35% Decaglycerol monolaurate

0.65% Span 20

75% 10 mM NaCl

For the preparation of the oil phase ethyl cellulose was dissolved in toluene. Decaglycerol monolaurate and Span 20 were added to the solution. The aqueous phase (10 mM NaCl) was added in the same manner as described in Example 1. The conductivity change of the system was monitored as described in Example 1. A nanoemulsion was formed comprising ethyl cellulose dissolved in toluene as the dispersed oil phase. The average droplet size of the emulsion was 122 nm.

Toluene was evaporated using a rotor stator evaporator, under reduced pressure, at 40° C., resulting in the formation of ethyl cellulose dispersion. The average size of the ethyl cellulose nanoparticles was 85 nm (z-average).

### Example 5

### Nanoparticles of Ethyl Cellulose and Nile Red

Nanoparticles of Ethyl cellulose and a fluorescent marker (Nile red), as an example of an active component, were prepared, using the following specific composition:

### Composition:

19.2% toluene containing 10⁻³ M Nile red

0.8% Ethyl cellulose (Ethyl cellulose from Sigma, having a viscosity of a 5% solution in toluene:ethanol 4:1 at 25° C.˜45 cPs)

4.35% Decaglycerol monolaurate

0.65% Span 20

75% 10 mM NaCl

For the preparation of the oil phase Nile red and ethyl cellulose were dissolved in toluene. Decaglycerol monolaurate and Span 20 were added to the solution. The aqueous phase (10 MM NaCl) was added in the same manner as described in Example 1. The conductivity change of the system was monitored as described in Example 1. A nanoemulsion was formed comprising of Nile red and ethyl cellulose dissolved in toluene as the dispersed oil phase. The average droplet size of the emulsion was 126 nm.

Toluene was evaporated using a rotor stator evaporator, resulting in the formation of ethyl cellulose and Nile red dispersion. The average size of the nanoparticles was 84 nm. The nanoparticles gave red fluorescence under exposure at wavelength of 365 nm.

### Example 6

### Nanoparticles of Ethyl Cellulose and Butylated Hydroxytoluene (BHT)

Nanoparticles of Ethyl cellulose and an antioxidant (Butylated Hydroxytoluene—BHT) as an example of an active component were prepared, using the following specific composition:

### Composition:

17.6% Toluene

0.4% EC

2% BHT

4.35% Decaglycerol monolaurate

0.65% Span 20

75% 10 mM NaCl

For the preparation of the oil phase BHT and ethyl cellulose were dissolved in toluene. Decaglycerol monolaurate and Span 20 were added to the solution. The aqueous phase (10 mM NaCl) was added in the same manner as described in example 1. The conductivity change of the system was monitored as described in example 1. A nanoemulsion was formed comprising of BHT and ethyl cellulose dissolved in toluene as the dispersed oil phase. The average droplet size of the emulsion was 118 nm.

Toluene was evaporated using a rotor stator evaporator, resulting in the formation of ethyl cellulose and Nile red dispersion. The average size of the nanoparticles was 88 nm.

### Example 7

### Nanoparticles of Lauryl Acrylate and Decaethylene Glycol Oleyl Ether (Brij 96v)

The present example describes formation of a nanoemulsion of a liquid monomer, obtained by phase inversion at constant temperature, using the following specific composition:

### Composition.

20% lauryl acrylate

7% Brij 96V

73% 10 mM NaCl

Specifically, the above amounts of Brij 96V and lauryl acrylate were mixed. The aqueous phase (10 mM NaCl) was added in the same manner as described in example 1. The conductivity change of the system was monitored as described in example 1. The result of the process is an emulsion having an average droplet size of 300 nm. This emulsion is further polymerized to form nanoparticles.

### Example 8

### Nanoparticles of Lauryl Acrylate and Decaethylene Glycol Oleyl Ether (Brij 96v)

The present example describes formation of polymeric nanoparticles, obtained by polymerization of nanodroplets and heating to a point of inversion (PIT). Nanoemulsions with the following composition were prepared:

### Composition:

20% lauryl acrylate

7% Brij 96V

73% 10 mM NaCl

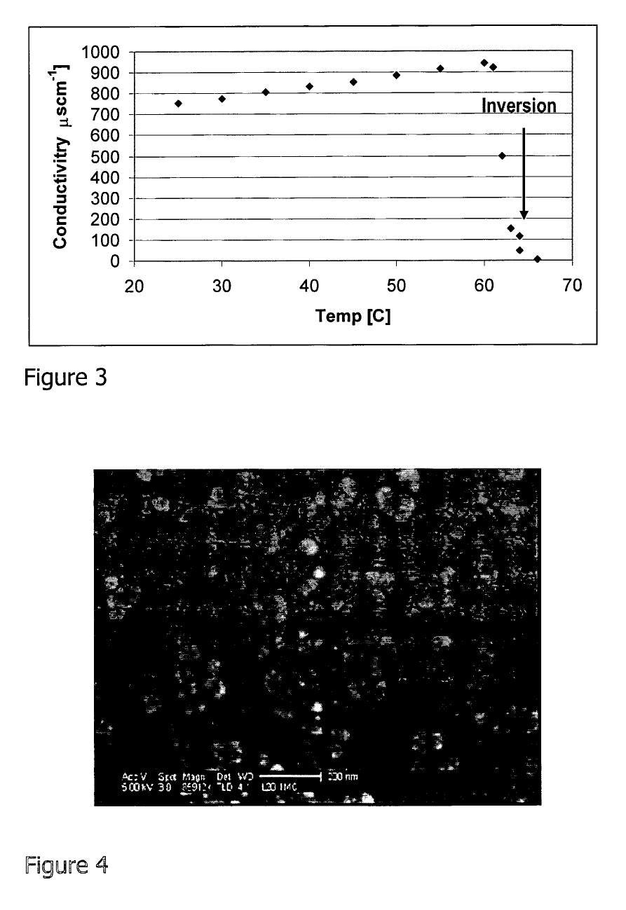

Specifically, crude emulsion was prepared using 20% lauryl acrylate and 7% Brij 96V, by using Ultra-Turrax homogenizer for 5 min, at arate of 8000 min⁻¹. Brij 96V was dissolved in the aqueous phase prior to the emulsification. The emulsion had an average droplet size of 360 nm with very high polydispersity. The emulsion was then heated to the point of inversion (PIT). The PIT was found by monitoring the conductivity of the emulsion during the heating process. FIG. 3 displays the change in conductivity during the heating process.

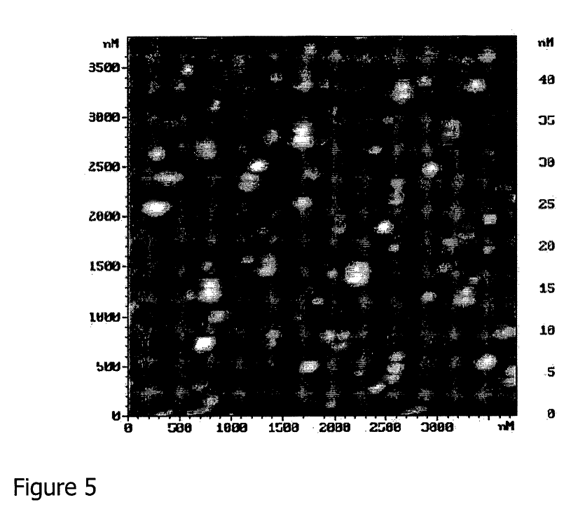

The heating was stopped at the point where the conductivity reached a value of zero. The hot emulsion was rapidly cooled in an ice bath. The PIT of this system was found to be 59° C. The result of this process was a nanoemulsion having an average droplet size of 72 nm. The polymerization of the oil droplets was carried out using a water soluble initiator, Ammonium persulfate (APS) activated by Ferrous ion, Fe⁺². Styrene was added to the reaction vessel in order to form a hydrophobic radical that can enter the oil droplets. 0.33 ml of 10% APS solution, 1.45 ml of 0.1M FeSO4 and 160 μl styrene were added to 20 gr of the nano-emulsion. The reaction was carried out for 4 hours at 40÷C. After 2 hours additional dose of 1.45 ml of 0.1M FeSO₄ and 0.33 ml of 10% APS solution was added. The resulting polymeric particles had an average diameter of 40 nm. FIG. 4 shows a SEM image of poly-lauryl acrylate nanoparticles obtained by the above method and FIG. 5 shows an atomic force microscope (AFM)image of the nanoparticles.

### Example 9

### Formation of Polymeric Nanoparticles

The example describes formation of polymeric nanoparticles, obtained by polymerization of nanodroplets, obtained by PIT. The nanoparticles contained a fluorescent marker (Pyrene), as an example of an active component.

The following composition was used:

### Composition:

20% of 0.5% pyrene in lauryl acrylate

7% Brij 96V

73% 10 mM NaCl

Specifically, Pyrene was dissolved in lauryl acrylate. Brij 96 was dissolved in the aqueous phase. The two phases were homogenized using an Ultra-Turrax homogenizer for 5 min, at a rate of 8000 min⁻¹ to form a crude emulsion. The emulsion was then heated to the point of inversion (PIT). The PIT was found by monitoring the conductivity of the emulsion during the heating process as described in example 8.

The heating was stopped at the point where the conductivity reached a value of zero. The hot emulsion was rapidly cooled in an ice bath. The PIT of this system was found to be 65° C. The result of this process was a nanoemulsion having an average droplet size of 106 nm. The polymerization was carried out as described in example 7. The resulting polymeric particles had an average diameter of 80 nm.

While this invention has been shown and described with reference to preferred embodiments thereof, it will be understood by those skilled in the art that many alternatives, modifications and variations may be made thereto without departing from the spirit and scope of the invention. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference.

1-65. (canceled)

66. A method for the production of nanoparticles of an active agent, the method comprising:
(a) mixing the active agent with a volatile solvent and at least one non ionic surfactant;
(b) adding to the mixture of (a) an aqueous phase to form a water-in-oil emulsion;
(c) continuously adding to the emulsion of (b) water at a rate enabling phase inversion and formation of oil-in-water nanoemulsion;
(d) evaporating the volatile solvent from the oil-in-water nanoemulsion of (c) to obtain nanoparticles of the active ingredient.

67. The method of claim 66 wherein the non-ionic surfactant in step (a) have HLB value in the range of 10-20.

68. The method of claim 66 wherein said non-ionic surfactant is selected from polyethoxylated sorbitan esters, polyglycerol esters, sucrose esters, ethoxylated alcohols, octylphenol ethoxylated, and mixtures of any of the above.

69. The method of claim 66 wherein the mixture of step (a) further comprises an additional ionic surfactant.

70. The method of claim 66 further comprising an additional step after step (d) selected from spray-drying or lyophilization thereby forming a powder of nanoparticles.

71. A method for the production of nanoparticles of an active agent, the method comprising:
(a) mixing the active agent with liquid monomers capable of polymerizing, and at least one non ionic surfactant;
(b) adding to the mixture of (a) an aqueous phase to form a water-in-oil emulsion;
(c) continuously adding to the emulsion of (b) water at a rate enabling phase inversion and formation of oil-in-water nanoemulsion;
(d) applying conditions enabling polymerization of the liquid monomer in the oil-in-water nanoemulsion of
(c) to obtain nanoparticles of the active ingredient.

72. The method of claim 71 wherein the monomers are selected from sterene, lauryl acrylate, stearyl acrylate, isodecyl acrylate, isooctyl acrylate, isotridecyl acrylate, isobornyl acrylate, lauryl methacrylate, lauryl methacrylate, stearyl methacrylate, isobornylmethacrylate, and mixtures of any of the above.

73. The method of claim 71 wherein the non-ionic surfactant is selected from polyethoxylated sorbitan esters, polyglycerol esters, sucrose esters, ethoxylated alcohols, octylphenol ethoxylated, and mixtures of any of the above.

74. The method of claim 71 wherein the non-ionic surfactant in step (a) have HLB value in the range of 10-20.

75. The method of claim 71 further comprising adding an initiator to the mixture of step (a) or immediately prior to step (d).

76. The method of claim 71 wherein the initiator is selected from a thermal initiator and a UV activated initiator.

77. The method of claim 76 wherein the initiator is a thermal initiator and the condition in step (d) is applying suitable temperatures.

78. The method of claim 76 wherein the initiator is a UV activated initiator and the condition in step (d) is applying UV radiation.

79. The method of claim 76 wherein the thermal initiator is water soluble thermal initiator and is added in step (c).

80. The method of claim 75 wherein the initiator is hydrophobic and is added to the oil phase in step (a).

81. The method of claim 75 wherein the initiator is hydrophilic and is added to the water phase immediately prior to step (d).

82. The method of claim 71 wherein the conditions in step (d) are selected from applying suitable temperature and applying UV radiation.

83. The method of claim 71 further comprising adding an activator in step (a).

84. The method of claim 71 further comprising adding an activator immediately prior to step (d).

85. The method of claim 71 further comprising an additional step after step (d) selected from spray-drying or lyophilization thereby forming a powder of nanoparticles.

86. The method of claim 71 wherein two different monomers are used, a first monomer being in the oily phase is added in step (a) and a second monomer is added in steps (b) and (c) to the aqueous phase.

87. The method of claim 86 wherein nanoencapsulation takes place in the interface between the first and second monomers during their polymerization.

88. A method for the production of nanoparticles of an active agent, the method comprising:
(a) mixing the active agent with a volatile solvent, at least one nonionic surfactant and an aqueous phase, to obtain a crude oil-in-water emulsion;
(b) raising the temperature of the crude oil-in-water emulsion of (a) to a phase inversion temperature (PIT) to obtain, a water-in-oil emulsion;
(c) cooling the water-in-oil emulsion of step (b) to obtain an oil-in-water nanoemulsion;
(d) evaporating volatile solvent from the oil in water nanoemulsion of (c) at a temperature below the PIT, to obtain nanoparticles of the active ingredient.

89. The method of claim 88 wherein the non-ionic surfactant in step (a) have HLB in the range of 10-20.

90. The method of claim 88 wherein said non-ionic surfactant is selected from polyethoxylated sorbitan esters, polyglycerol esters, sucrose esters, ethoxylated alcohols, octylphenol ethoxylated, and mixtures of any of the above.

91. The method of claim 88 wherein the mixture of step (a) further comprises an additional ionic surfactant.

92. The method of claim 91 wherein said ionic surfactant is sodium dodecyl sulphate.

93. The method of claim 88 wherein the raise to the PIT temperature, in step (b), is gradual.

94. The method of claim 88 wherein the cooling in step (c) is rapid cooling in order to stabilize the nanoemulsion obtained during the inversion.

95. The method of claim 88 wherein the evaporation in step (d) is done at a temperature below the PIT.

96. The method of claim 88 further comprising an additional step after step (d) selected from spray drying or lyophilization thereby forming a powder of nanoparticles.

97. The method of claim 88 wherein evaporation in step (d) is carried out simultaneously with spray drying or lyophilization converting the nanoparticles formed by evaporation into powder of nanoparticles.

98. A method for the production of nanoparticles of an active ingredient, the method comprising:
(a) mixing the active agent with liquid monomers capable of polymerizing, at least one nonionic surfactant and an aqueous phase to obtain a crude oil-in-water emulsion;
(b) raising the temperature of the crude oil-in-water emulsion of (a) to a phase inversion temperature (PIT) to obtain, a water-in-oil emulsion;
(c) cooling the water-in-oil emulsion of step (b) to obtain an oil-in-water nanoemulsion;
(d) providing conditions enabling polymerization, and which do not cause a raise of a temperature to a temperature above the PIT temperature, to the oil-in-water nanoemulsion of (c) to obtain nanoparticles of the active ingredient.

99. The method of claim 98 wherein the monomers are selected from sterene, lauryl acrylate, stearyl acrylate, isodecyl acrylate, isooctyl acrylate, isotridecyl acrylate, isobornyl acrylate, lauryl methacrylate, lauryl methacrylate, stearyl methacrylate, isobornylmethacrylate, and mixtures of any of the above.

100. The method of claim 98 wherein the non-ionic surfactant is selected from polyethoxylated sorbitan esters, polyglycerol esters, sucrose esters, ethoxylated alcohols, octylphenol ethoxylated, and mixtures of any of the above.

101. The method of claim 98 wherein the non-ionic surfactant in step (a) have HLB value in the range of 10-20.

102. The method of claim 98 further comprising adding an initiator.

103. The method of claim 102 wherein said initiator is selected from thermal initiator and UV activated initiator.

104. The method of claim 102 wherein said initiator is a thermal initiator and is added after phase inversion raise in temperature in step (b).

105. The method of claim 103 wherein the thermal initiator is added between steps (c) and (d) and the condition in step (d) is to raise the temperature to a temperature lower than PIT.

106. The method of claim 103 wherein the thermal activated initiator is hydrophilic and is added to the water phase of the oil-in-water nanoemulasion obtained by step (c).

107. The method of claim 103 wherein the UV activated initiator is hydrophobic and is added to the oil phase of the mixture in step (a).

108. The method of claim 103 wherein the UV activated initiator is hydrophilic and is added to the aqueous phase in step (a).

109. The method of claim 103 wherein the UV activated initiator is hydrophilic and is added to the water in the oil-in-water nanoemulsion of step (c).

110. The method of claim 103 wherein said initiator is UV initiator and the condition of step (d) is application of UV radiation sufficient to begin polymerization.

111. The method of claim 102 further comprising adding an activator.

112. The method of claim 108 wherein said activator is thermal activator and is selected from transition metal ions.

113. The method of claim 111 wherein said activator is added in step (a) to the oil phase or aqueous phase.

114. The method of claim 111 wherein said activator is added in step (c) to the water phase.

115. The method of claim 98 wherein two different monomers are used, a first monomer is being in the oily phase and a second monomer is dissolved in the aqueous phase, said first and second monomers are added in step (a).

116. The method of claim 115 wherein nanoencapsulation takes place in the interface between the first and second monomers during their polymerization.
